(19) United States
(12) Patent Application Publication
(10) Pub. No.: US 2007/0233138 A1
(43) Pub. Date: Oct. 4, 2007

(54) Apparatuses and methods for arthroplastic surgery

(75) Inventors: Marvin Figueroa, Warsaw, IN (US); Toby N. Farling, Warsaw, IN (US); Adam M. Griner, Columbia City, IN (US); Vinod Ponmola, Warsaw, IN (US); Marlowe Goble, Cokeville, WY (US)

Correspondence Address: ZIMMER TECHNOLOGY - BAKER & DANIELS, 111 EAST WAYNE STREET, SUITE 800, FORT WAYNE, IN, 46802, US

(73) Assignee: Zimmer Technology, Inc.

(21) Appl. No.: 11/477,243

(22) Filed: Jun. 29, 2006

Related U.S. Application Data
(63) Continuation-in-part of application No. 11/342,357, filed on Jan. 27, 2006.

(51) Int. Cl. A61F 5/00

(52) U.S. Cl. 606/87

(57) ABSTRACT

A distal cut guide assembly including an intramedullary rod, an alignment guide extending from the rod at an angle and having a slot, a cut guide body having a rail defining a rail axis, a coupling member slidably coupled to rail, and a handle slidably and pivotally joining coupling member to slot and defining a handle axis. The position of the guide body being adjustable relative to the bone along rail axis and slot and about handle axis. A tibial cut guide assembly including a cut guide support member and a cut guide pivotally attached to the support member. An arthroplastic spacer including a spacer block having an axis of symmetry. The spacer includes a handle having a linear portion aligned with and longitudinally bisected by the axis of symmetry and a curved portion coupling linear portion to block. The curved portion is spaced from the axis of symmetry.

### PRIORITY REFERENCE

This application is a continuation-in-part of, and claims priority under 35 U.S.C. §120 to, U.S. application Ser. No. 11/342,357, entitled APPARATUSES AND METHODS FOR ARTHROPLASTIC SURGERY and filed on Jan. 27, 2006 in the names of Toby N. Farling et al.

### BACKGROUND

The present invention relates to apparatuses and methods for arthroplastic surgery and, more particularly, to cut guide apparatuses for resecting the end of a bone and spacer apparatuses for measuring the joint space between resected bones.

Orthopedic procedures for the replacement of all, or a portion of, a patient's joint typically require resecting (cutting) and reshaping of the ends of the bones of the joint. For instance, total knee replacement procedures typically involve resecting the distal end of the femur and the proximal end of the tibia prior to implanting the prosthesis components. Resecting the distal end of the femur often involves making several cuts of the distal end of the femur including a distal cut. Resecting the proximal end of the femur often involves making a proximal cut.

Cut guides have been developed to guide the saw and achieve the proper angle and position of these cuts. Conventional cut guides are often in the form of blocks having slots therein for receiving and guiding the saw. In use, the block is positioned against the bone with the help of positioning and alignment equipment. The block is then secured to the bone using fasteners. For instance, in the case of certain known distal cut guides used for resecting the end of the femur, the cut guide block is slidably mounted to an alignment guide, which is mounted at an angle to a intramedullary rod, as shown in U.S. Patent Publication No. 2004/0153066 to Coon et al. The intramedullary rod is inserted into a pre-drilled hole in the intramedullary canal of the femur such that the alignment guide extends across the distal end of the femur and cut guide block is positioned proximal the side of the femur. The cut guide block may be slid toward or away (medially-laterally) from the femur until it is properly positioned against the surface of the femur. The block is then fixed to the bone using fasteners. The intramedullary rod and alignment guide are removed and a saw is inserted through the slot to resect the distal end of the femur. Although effective in guiding the cutting of the femur, it may be challenging to align the block (and the slot) anteriorly-posteriorly. In addition, it may also be a challenge to position the block against the bone in cases where the surface of the bone is irregular. Similar challenges are presented when attempting to position a cut guide block against the tibia bone.

Once the distal end of the femur and the proximal end of the tibia are resected, it is beneficial for the surgeon to measure the space or gap between the tibia and the femur to insure the space is suitable and the angle of the cuts are proper. This may involve inserting a spacer or other measurement device into the gap. The spacer typically includes a spacer block and a handle extending linearly and anteriorly from the spacer block. Conventional knee replacement procedures often involve everting (flipping over) the patella to create additional space in which cut blocks can access the knee and spacers can access the gap between the femur and the tibia. However, to minimize disruption to nearby tissue and shorten recovery time, minimally invasive surgical techniques are encouraged. Minimally invasive surgical techniques typically involve smaller incisions and tighter work spaces and avoid everting the patella.

Accordingly, there is a need for cut guides and spacers that can be more effectively positioned and used in minimally invasive techniques.

### SUMMARY

The present invention provides apparatuses and methods for arthroplastic surgery and, more particularly, to cut guide apparatuses for resecting the end of a bone and spacer apparatuses for measuring the joint space between resected bones.

In one form the invention provides a distal cut guide assembly for cutting a distal end of a femur. The distal cut guide assembly includes an intramedullary rod, an alignment guide extending from the rod, a cut guide body, a coupling member coupled to the cut guide body, and a handle coupling the coupling member to the alignment guide.

The intramedullary rod defines a rod axis and is configured to be inserted into the intramedullary canal of the femur. The alignment guide has an upper surface, an opposing lower surface and an elongated slot extending therethrough from the upper surface to the lower surface. The slot defines a slot axis extending at an angle relative to the rod axis. The cut guide body has opposing first and second surfaces, opposing distal and proximal sides extending between the first and second surfaces, and opposing anterior and posterior ends extending between both the first and second surfaces and the distal and proximal sides. The cut guide body defines at least one cut guide surface extending from the first surface to the second surface. The distal side has a rail extending between the anterior and posterior ends and defining a rail axis.

The coupling member has an upper portion and a lower portion. The upper portion is slidably received within the slot and has an opening extending therein. The opening is in alignment with the slot. The lower portion protrudes from the slot and has a channel. The rail of the cut guide body is slidably received within the channel such that the rail axis extends at an angle relative to the slot axis and the rod axis. The upper portion is at least partially rotatable within the slot to thereby adjust the angle of the rail axis relative to the slot axis. The handle has an engagement end extending through the slot and adjustably engaging with the opening of the coupling member. The handle is adjustable between a locked position wherein the engagement with the opening prevents both the upper portion from sliding and rotating within the slot and the rail from sliding within the channel, and a released position wherein the engagement with the opening allows both the upper portion to slide and rotate within the slot and the rail to slide within the channel.

In another form, the present invention provides a tibial cut guide assembly for cutting the proximal end of the tibia bone. The cut guide assembly includes a cut guide support member having an upper surface, a lower surface, and an opening extending between the upper and lower surfaces. The support member includes a stop post extending vertically from the upper surface. The cut guide assembly also includes a cut guide having a first bone engaging surface, an opposing second surface and opposing proximal and distal sides extending between the first and second surfaces. The cut guide has at least one cut guide surface extending between the first and second surfaces. The cut guide has a mounting post extending vertically from the distal side. The mounting post is rotatably received within the opening of the support member. The cut guide has a track defined in the distal side. The stop post is slidably disposed within the track and cooperates with the track to limit the rotation of the mounting post in the opening. The cut guide assembly also includes a vertical fixation member in engagement with the mounting post and the support member to prevent vertical movement of the mounting post within the opening.

In yet another form, the present invention provides an arthroplastic spacer for gauging a gap between the distal end of a femur and the proximal end of a tibia. The spacer includes a spacer block having a medial side, a lateral side opposite the medial side, an anterior side and a posterior side opposite the anterior side. The spacer block has a superior surface and an inferior surface opposite the superior surface. The inferior and superior surfaces extend between medial, lateral, anterior and posterior sides. The spacer block includes a perimeter surface extending between superior and inferior surfaces and wrapping both anteriorly-posteriorly and medially-laterally about the perimeter of the spacer block. The spacer block has a medial-lateral width extending between the medial and lateral sides and an axis of symmetry equally dividing the medial-lateral width. The spacer includes a handle having a linear portion and a curved portion. The curved portion has a first end extending from the perimeter surface at a point either medial or lateral to the axis of symmetry and a second end coupled to the linear portion. The linear portion is aligned with and longitudinally bisected by the axis of symmetry. The curved portion is spaced from the axis of symmetry.

In another aspect, the present invention provides a tibial cut guide assembly for cutting a proximal end of the tibia bone. The tibia bone defines a tibial axis and includes the proximal end and an anterior surface. The cut guide assembly comprises a cut guide support member having a first end and a second end. The first end has an upper surface and a lower surface. The support member has an opening extending through the first end from the upper surface to the lower surface. The opening defines a first axis and the support member has a hole extending into the first end and intersecting the opening. The cut guide assembly further includes a cut guide having a first bone engaging surface, an opposing second surface, and opposing proximal and distal sides extending between the first and second surfaces. The cut guide has at least one cut guide surface extending from the first surface to the second surface. The cut guide has a mounting post extending vertically from the distal side. The mounting post has a sidewall extending about the first axis and a notch defined in and extending about a portion of the sidewall. The mounting post is rotatably received within the opening of the support member such that the notch is aligned with the hole. The pin extends through the hole and has a first end extending into the notch to prevent vertical movement of the mounting post along the first axis. The first end of the pin is limitedly pivotal within the notch to permit and limit rotation of the mounting post about the first axis.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is a side view of a cut guide body and coupling member of a distal cut guide assembly according to one embodiment of the present invention;

FIG. 2 is a top perspective view of the cut guide body and coupling member of FIG. 1;

FIG. 3 is a top view of the cut guide body and coupling member of FIG. 1;

FIG. 4 is a perspective view of the coupling member of FIG. 1;

FIG. 5 is a perspective view of a distal cut guide assembly according to one embodiment of the present invention;

FIG. 6 is an exploded view of the distal cut guide assembly of FIG. 5;

FIG. 7 is another perspective view of the distal cut guide assembly of FIG. 5;

FIG. 8 is an anterior view of a femur with the cut guide assembly of FIG. 5 mounted thereon;

FIG. 9 is a medial view of the femur of FIG. 8;

FIG. 10 is a sectional view of the distal cut guide assembly of FIG. 7 taken along lines 10-10;

FIG. 11 is a perspective view of a cut guide body according to another embodiment of the present invention;

FIG. 12 is a bottom view of the cut guide body of FIG. 11;

FIG. 13 is another perspective view of the cut guide body of FIG. 11;

FIG. 14 is a perspective view of a tibial cut guide assembly according to one embodiment of the present invention;

FIG. 15 is a front view of the tibial cut guide assembly of FIG. 14;

FIG. 15A is a sectional view of the tibial cut guide assembly of FIG. 15 taken along lines 15A-15A;

FIG. 16 is a top view of the tibial cut guide assembly of FIG. 14;

FIG. 17 is an exploded view of the tibial cut guide assembly of FIG. 14;

FIG. 18 is a bottom perspective view of the tibial cut guide of the cut guide assembly of FIG. 14;

FIG. 19 is an exploded view of a tibial cut guide assembly according to another embodiment of the present invention;

FIG. 20 is a perspective view of an arthroplastic spacer in accordance with one embodiment of the present invention;

FIG. 21 is a bottom (inferior) view of the arthroplastic spacer of FIG. 20;

FIG. 22 is a side (lateral) view of the arthroplastic spacer of FIG. 20;

FIG. 23 is a top (superior) view of the arthroplastic spacer of FIG. 20 being inserted into the knee joint atop the proximal end of a tibia;

FIG. 24 is a top (superior) view of the arthroplastic spacer of FIG. 20 in position atop the proximal end of the tibia;

FIG. 25 is a side (lateral) view of the knee joint with the arthroplastic spacer of FIG. 20 in position in the joint space;

FIG. 26 is a front (anterior) view of a tibial cut guide assembly according to another embodiment of the present invention positioned against a tibia bone;

FIG. 27 is a perspective view of the cut guide support member of the assembly of FIG. 26;

FIG. 28 is a bottom (inferior) view of the support member of FIG. 27;

FIG. 29 is a side (lateral) view of the support member of FIG. 27;

FIG. 30 is a side (lateral) view of the cut guide of the assembly of FIG. 26;

FIG. 31 is a back (posterior) view of the cut guide of FIG. 30;

FIG. 32 is a front (anterior) view of an assembly of the support member and cut guide of FIGS. 27 and 30;

FIG. 32A is a sectional view of the assembly of FIG. 32 taken along line 32A-32A;

FIG. 33 is a perspective view of the assembly of FIG. 32;

FIG. 34 is an exploded view of the assembly of FIG. 26;

FIG. 35 is a perspective view of the vertical fixation pin of the assembly of FIG. 34; and

FIG. 36 is a perspective view of the assembly of FIG. 26.

Corresponding reference characters indicate corresponding parts throughout the several views. Although the drawings represent embodiments of the present invention, the drawings are not necessarily to scale and certain features may be exaggerated in order to better illustrate and explain the present invention. Although the exemplification set out herein illustrates embodiments of the invention, in several forms, the embodiments disclosed below are not intended to be exhaustive or to be construed as limiting the scope of the invention to the precise forms disclosed.

### DETAILED DESCRIPTION

The embodiments hereinafter disclosed are not intended to be exhaustive or limit the invention to the precise forms disclosed in the following description. Rather the embodiments are chosen and described so that others skilled in the art may utilize its teachings.

The present invention will now be described with reference to the attached figures. The description below may include references to the following terms: anterior (at or near the front of the body, as opposed to the back of the body); posterior (at or near the back of the body, as opposed to the front of the body); lateral (at or near the side of the body, farther from the midsagittal plane, as opposed to medial); medial (at or near the middle of the body, at or near the midsagittal plane, as opposed to lateral); proximal (toward the beginning, at or near the head of the body, as opposed to distal) and distal (further from the beginning, at or near the foot of the body, as opposed to proximal).

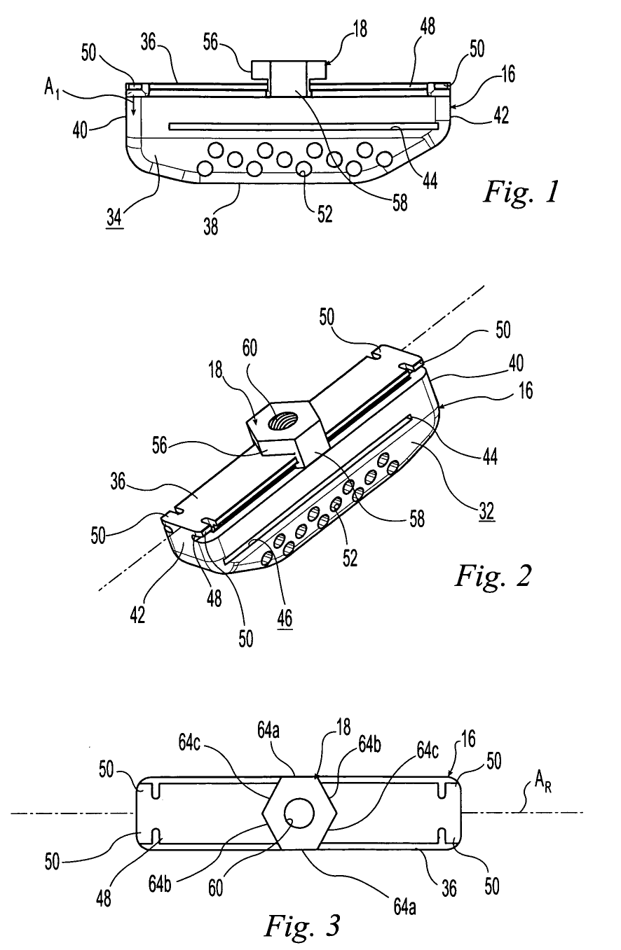

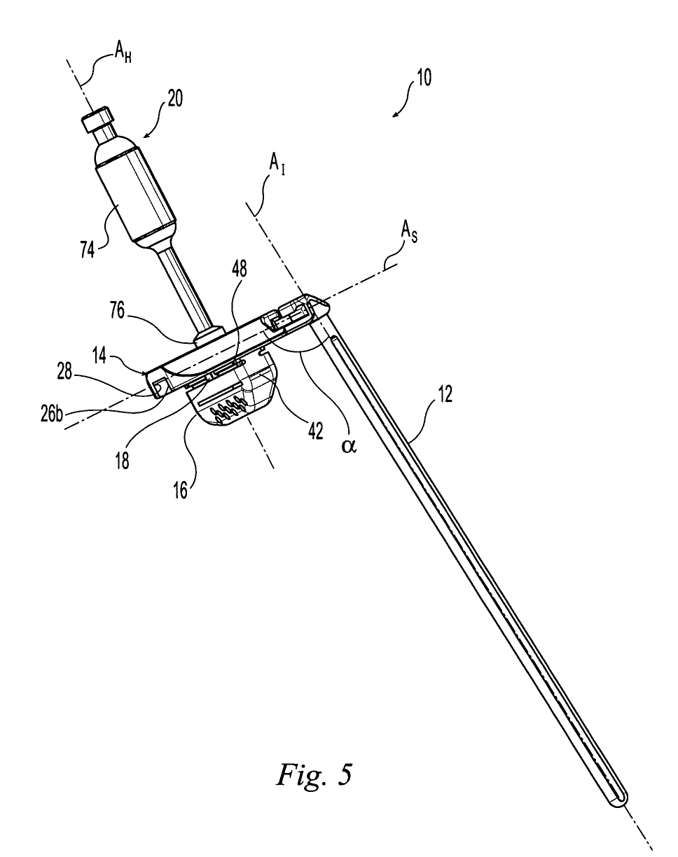

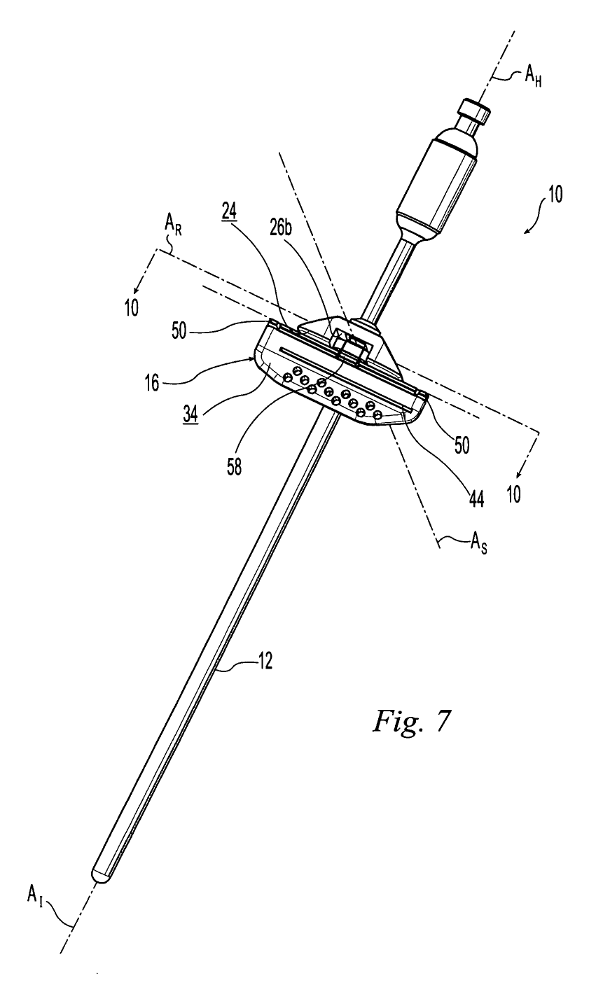

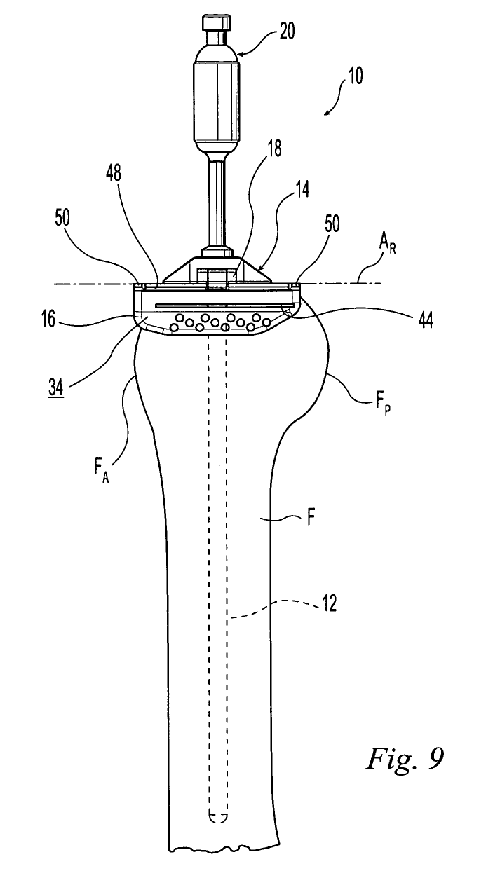

Referring first to FIGS. 1-10, distal cut guide assembly 10 according to one embodiment of the present invention will now be described. As is described in further detail below, distal cut guide assembly 10 is used to prepare (i.e. resect) the distal end of a femur. As illustrated in FIGS. 5-7, distal cut guide assembly 10 generally includes intramedullary “IM” rod 12, alignment guide 14 coupled to and extending from IM rod 12, cut guide body 16, coupling member 18 slidably coupled to guide body 16, and handle 20 extending through alignment guide 14 and coupled to coupling member 18.

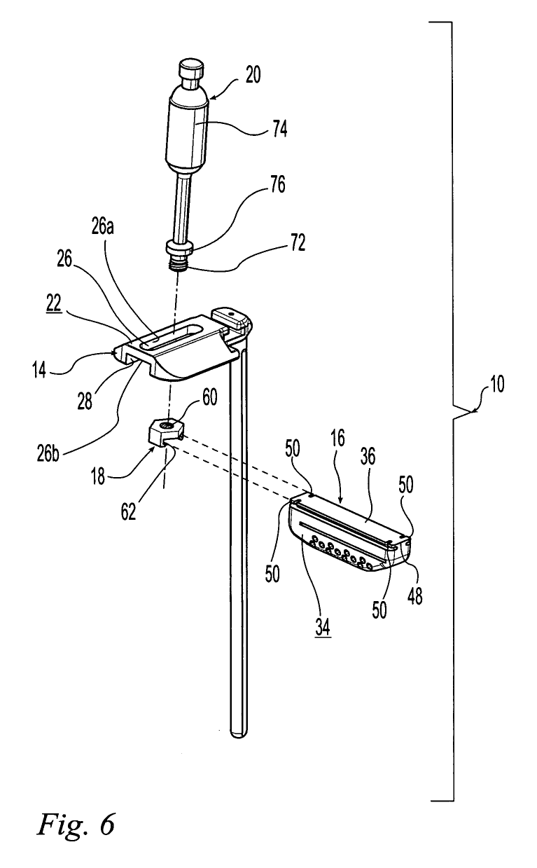

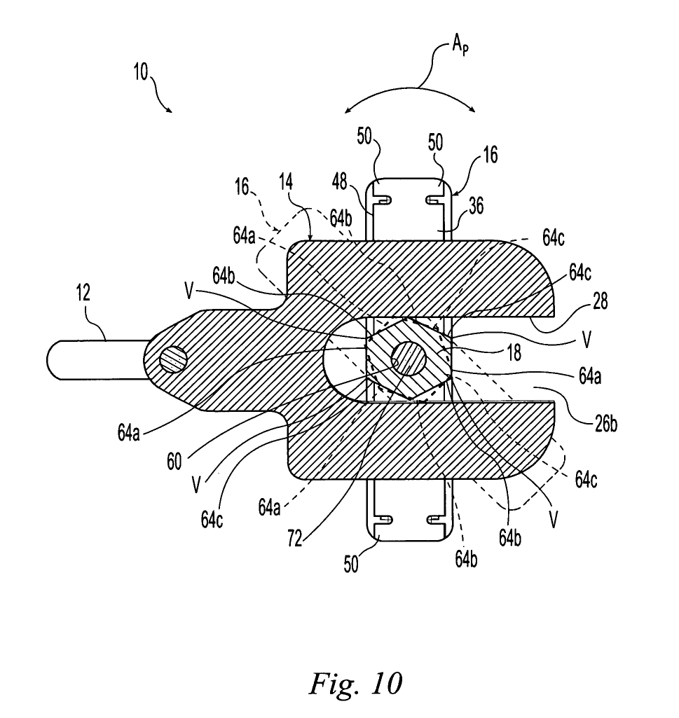

Referring still to FIGS. 5-7, IM rod 12 is elongate and defines IM rod axis A_{I}. Alignment guide 14 is attached to IM rod 12 and extends from an end of IM rod 12. The assembly of IM rod 12 and alignment guide 14 may be integrally formed as a single unit. Alternatively, IM rod 12 and alignment guide 14 may be two separate components attachable to one another. Alignment guide 14 includes upper surface 22, lower surface 24, and slot 26 extending through alignment guide 14 from upper surface 22 to lower surface 24. Slot 26 extends along slot axis A_{S}. As noted above, alignment guide 14 extends from IM rod 12. More particularly, alignment guide 14 extends from IM rod 12 such that slot axis A_{S} forms angle α relative to IM rod axis A_{I}. Angle α may vary to accommodate the differences between the anatomic axis (an imaginary line drawn down the center of the femoral canal) and the mechanical axis (a line passing through the center of the hip, the center of the knee and the center of the ankle), which commonly varies from between 4° to 8°. For instance, as described in U.S. Publication No. 2004/0153066 to Coon et al. filed as U.S. patent application Ser. No. 10/357,282 entitled Apparatus for Knee Surgery and Method of Use, assigned to the assignee of the present invention and hereby incorporated by reference, the differences between the anatomic and mechanical axes may be suitably represented at 4°, 6° or 8°. Accordingly, to accommodate this difference, angle α may be 94°, 96° or 98°. Of course, distal cut guide assembly 10 may be configured such that angle α accommodates any particular difference between the anatomic and mechanical axes. As illustrated in FIG. 6, slot 26 includes upper, captured portion 26a proximal upper surface 22 and lower, open-ended portion 26b proximal lower surface 24. Open-ended portion 26b is defined, at least in part, by pair of parallel sidewalls 28, as shown in FIGS. 6 and 10.

Turning now to FIGS. 1-3, cut guide body 16 includes first bone-engaging surface 32 and second surface 34 opposite bone-engaging surface 32. Opposing distal and proximal sides 36, 38 extend between first and second surfaces 32, 34, while opposing anterior and posterior ends 40, 42 extend between both first and second surfaces 32, 34 and distal and proximal sides 36, 38. Cut guide body 16 includes elongated cut guide aperture 44 extending through cut guide from first surface 32 to second surface 34. Cut guide aperture 44 is adapted to receive a cutting instrument such as a saw and provides cut guide surface 46, which guides the saw in cutting the distal end of the femur. A plurality of fastener receiving holes 52 extend through cut guide body 16 from first surface 32 to second surface 34. Fastener receiving holes 52 are adapted to receive fasteners (not shown) such as pins, screws or nails, which are used to secure cut guide body 16 to the femur.

Referring still to FIGS. 1-3, distal side 36 of cut guide body 16 includes rail 48 in the form of a T-shaped projection extending between anterior and posterior ends 40, 42 and defining rail axis A_{R}. Distal side 36 also includes pair of locking tabs 50 at each end of rail 48 proximal anterior and posterior ends 40, 42. As discussed in further detail below, locking tabs 50 are adapted to be deflected or bent downward away from rail 48.

Turning now to FIGS. 1-4 and 6, coupling member 18 has a form similar to that of a hexagonal nut. Coupling member 18 has upper portion 56 and lower portion 58. Threaded opening 60 extends through coupling member 18 from upper portion 56 to lower portion 58. As illustrated in FIGS. 6 and 10, upper portion 56 is slidably received in lower, open-ended portion 26b of slot 26 of alignment guide 14. Referring to FIGS. 1-4 and 7, lower portion 58 protrudes from slot 26 and includes T-shaped channel 62, which is complementary to and slidably receives rail 48 of cut guide body 16. As previously mentioned, coupling member 18 has a shape similar to that of a hexagonal nut. As illustrated in FIGS. 2-4 and 10, coupling member 18 has a hexagonal (six-sided) cross-section and includes three pair of opposing walls 64a, 64b, 64c. Each of pair of opposing walls 64a has a length that is less than the length of each of pairs 64b, 64c. This configuration allows upper portion 56 of coupling member 18 to be at least partially rotatable within slot 26. More particularly, the unequal lengths of walls 64a relative to walls 64b-c allow coupling member 18 to rotate in either a clockwise or a counterclockwise direction until one of vertices V between wall 64a and an adjacent wall 64b or 64c contacts sidewall 28 of slot 26b. At this point, further rotation is prohibited. The dashed lines in FIG. 10 indicate the freedom of movement in the counterclockwise direction.

Turning now to FIGS. 5-7, handle 20 is elongate and extends between first engagement end 72 and second gripping end 74. Engagement end 72 is threaded and is configured to extend through slot 26 of alignment guide 14 and threadingly engage opening 60 of coupling member 18. Handle 20 includes collar 76 adjacent engagement end 72. As is discussed in further detail below, collar 76 is sized and configured so as not to pass through slot 26 but rather to bear against upper surface 22 of alignment guide 14 proximal slot 26. Handle 20 defines handle axis A_{H} extending along the length of handle 20. Due to angle α between IM rod axis A_{I} and slot axis A_{S}, when cut guide assembly 10 is assembled, handle axis A_{H} extends relative to IM rod axis A_{I} at an angle equal to the difference between the anatomic axis and mechanical axis (i.e. 4°, 6°, 8° or any other predetermined angle).

Turning now to FIG. 6, the process of assembling distal cut guide assembly 10 will now be described. Although the process described below is set forth in a particular series of assembly steps, these steps may be performed in alternative orders. First, T-shaped rail 48 of cut guide body 16 is slid into complementary T-shaped channel 62 of coupling member 18 to slidably couple cut guide body 16 to coupling member 18. Coupling member 18 is now slidable along rail axis A_{R} (FIGS. 2, 3 and 7). To prevent rail 48 from sliding out of channel 62, locking tabs 50 adjacent anterior and posterior ends 40, 42 may be bent downward in the direction of arrow A₁ (FIG. 1). When bent downward locking tabs 50 block rail 48 proximal anterior and posterior ends 40, 42 thereby preventing rail 48 from disengaging from channel 62.

Referring still to FIG. 6, upper portion 56 of coupling member 18 is then positioned in lower, open ended portion 26b of slot 26. Engagement end 72 of handle 20 is inserted through captured portion 26a of slot 26 and into threaded opening 60 of coupling member 18. Handle 20 is rotated to threadingly, but loosely, engage engagement end 72 with opening 60, such that coupling member 18 is free to slide and rotate within slot 26. When assembled, the position of cut guide body 16, and thus cut guide surface 46, is adjustable in three directions. First, using handle 20, cut guide body may be moved along slot axis A_{S} toward or away from IM rod 12 by sliding handle 20 and coupling member 18 along slot 26. Captured portion 26a of slot 26 prevents handle 20, and therefore coupling member 18, from sliding out of slot 26. Second, cut guide body 16 is slidable along rail axis A_{R} by sliding rail 48 within channel 62. Thirdly, the angle of rail axis A_{R}, and thus cut guide surface 46 of cut guide body 16, relative to slot axis A_{S} may be adjusted by rotating coupling member 18 within slot 26 about handle axis A_{H}.

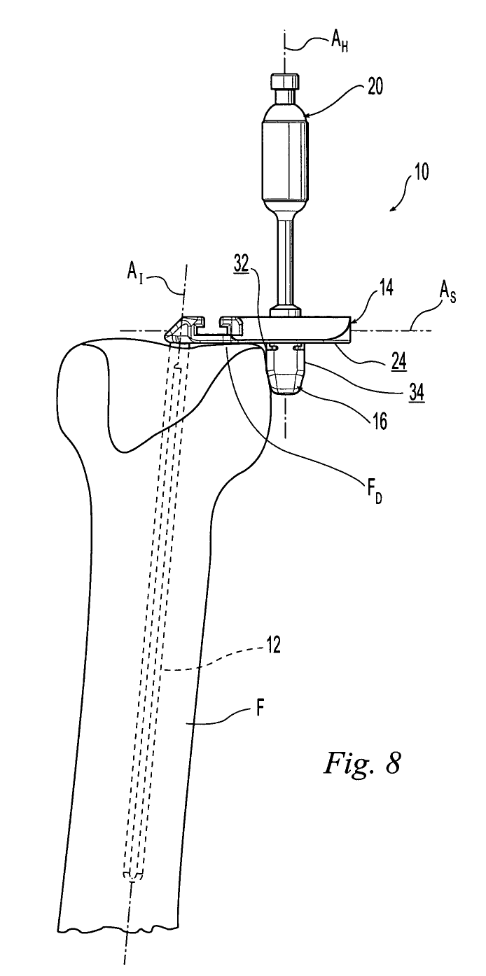

Turning now to FIGS. 6 and 8-10, use of distal cut guide assembly 10 will now be described. In use, an IM rod 12/alignment guide 14 assembly is selected having angle α corresponding to the difference between the patient's anatomic axis and mechanical axis. A hole (not shown) is drilled through the center of the patellar sulcus of femur F and into the intramedullary canal of femur F. Distal cut guide assembly 10 is assembled as described above and IM rod 12 is inserted into the pre-drilled hole using handle 20. IM rod 12 is inserted into the hole until lower surface 24 of alignment guide 14 contacts distal end F_{D} of femur F. Using handle 20, cut guide body 16 is slid along slot axis A_{S} toward femur F until first bone engaging surface 32 is adjacent femur F (FIG. 8). The angle of rail axis A_{R} (and bone engaging surface 32) relative to the surface of femur F may be adjusted by rotating coupling member 18 in either a clockwise or counterclockwise direction within slot 26 to accommodate variations in the surface of the femur and to better position cut guide body 16 against the femur F. Finally, cut guide body 16 is properly positioned between anterior side F_{A} and posterior side F_{P} of the distal end of femur F by sliding rail 48 within channel 62 along rail axis A_{R}. When cut guide body is properly positioned in all three directions, handle 20 is rotated thereby further engaging engagement end 72 with opening 60. When handle 20 is securely tightened, alignment guide 14 is clamped between collar 76 of handle 20 and coupling member 18 thereby locking coupling member 18 in position within slot 26 and preventing coupling member 18 from sliding or rotating within slot 26. In addition, when handle 20 is securely tightened, distal side 36 of cut guide body 16 is brought into firm, abutting engagement with lower surface 24 of alignment guide 14 thereby preventing rail 48 of cut guide body 16 from sliding in channel 62 of coupling member 18 and securing cut guide body 16 in position on rail axis A_{R}.

Once cut guide body 16 is secured in position, fasteners (not shown) such as pins or nails may be inserted through one or more fastener receiving holes 52 and into femur F as shown in U.S. Publication 2004/01153066 previously incorporated by reference herein. Once cut guide body 16 is secured to femur F with fasteners, handle 20 is rotated out of threaded engagement with coupling member 18 and handle 20 is removed. Next, IM rod 12 and alignment guide 14 are removed from femur F leaving cut guide body 16 fastened to femur F. A cutting instrument, such as a saw, may be inserted through cut guide aperture 44 and cut guide surface 46 is used to guide the saw in resecting the end of the femur.

Although in the exemplary embodiment described above, channel 62 and rail 48 are complementary T-shaped features, channel 62 and rail 48 may have any shape suitable to provide a sliding engagement between coupling member 18 and cut guide body 16. For instance, channel 62 and rail 48 may be dove-tail or semi-circular in shape. Furthermore, the female engagement feature (i.e. channel 62) and the male engagement member (i.e. rail 48) need not be defined on coupling member 18 and cut guide body 16, respectively. Rather, the female engagement member may be formed on cut guide body 16, while the male engagement member may be formed on coupling member 18.

Further, coupling member 18 is illustrated as having a hexagonal cross-sectional shape wherein vertices V between sides of unequal lengths serve to limit the rotational movement of coupling member 18 within slot 26. However, coupling member 18 may have alternative cross-sectional shapes such as circular, oval or other shapes. Coupling member 18 may incorporate other stop features, such as protrusions or bosses, to limit the rotational movement of coupling member 18 within slot 26. In addition, coupling member 18 may be configured to allow full rotation of coupling member 18 within slot 26.

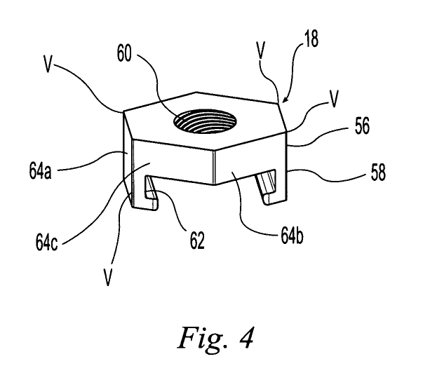

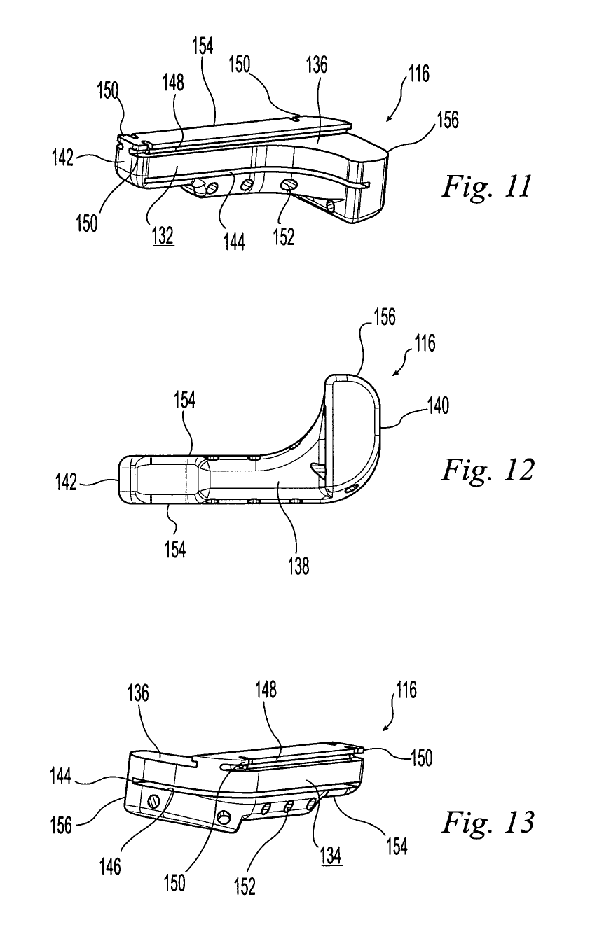

Turning now to FIGS. 11-13 an alternative cut guide body 116 for use in distal cut guide assembly 10 (FIG. 6) is illustrated. Cut guide body 116 includes first, bone-engaging surface 132 and opposite second surface 134. Opposing distal and proximal sides 136, 138 extend between first and second surfaces 132, 134, and opposing anterior and posterior ends 140, 142 extend between both first and second surfaces 132, 134 and distal and proximal sides 136, 138. Cut guide aperture 144 extends through cut guide body 116 from first surface 132 to second surface 134 and provides cut guide surface 146. Cut guide body 116 includes T-shaped rail 148 projecting from distal side 163. Rail 148 is configured to be slidably received within channel 62 of coupling body 18 (FIGS. 4 and 6) in the same manner as rail 48 of cut guide body 16 (FIGS. 1-3 and 6). Cut guide body 116 also includes locking tabs 150 on distal side 163 proximal anterior and posterior ends 40, 42. Locking tabs function in the same manner as locking tabs 50 (FIG. 1). As illustrated in FIGS. 11-13, cut guide body 116 is L-shaped and includes main body portion 154 and leg portion 156, which extends at an angle from main body portion 154. Cut guide aperture 144 and cut guide surface 146 extend through both main body portion 154 and leg portion 156.

In use, cut guide body 116 is assembled to coupling body 18, handle 20, alignment guide 14 and IM rod 12 (FIG. 6) in the same manner as cut guide body 16. Main body portion 154 is positioned against the side of the femur, and leg portion 156 extends laterally over the anterior side of the femur to provide guidance of the cut over a larger area.

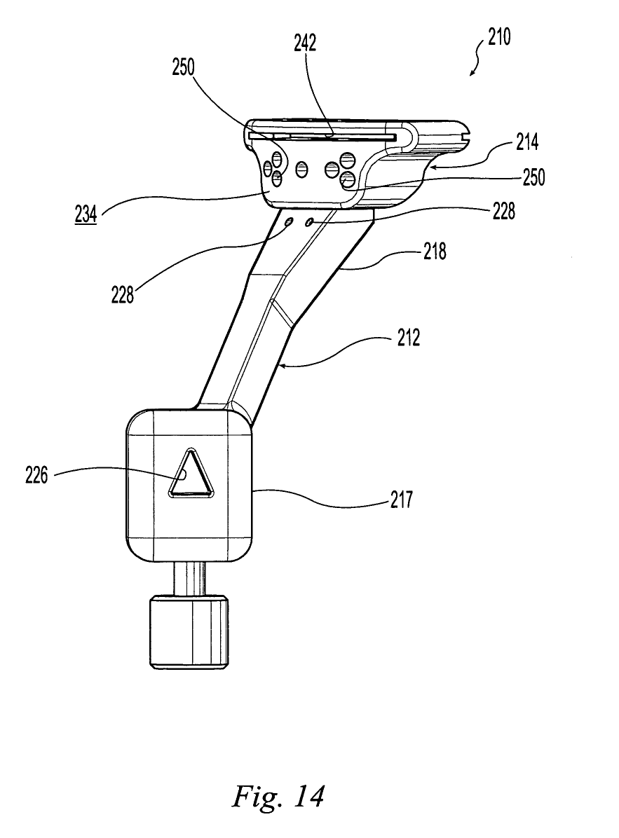

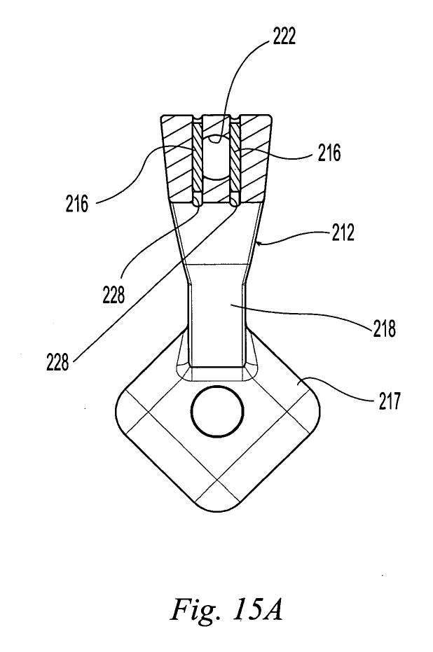

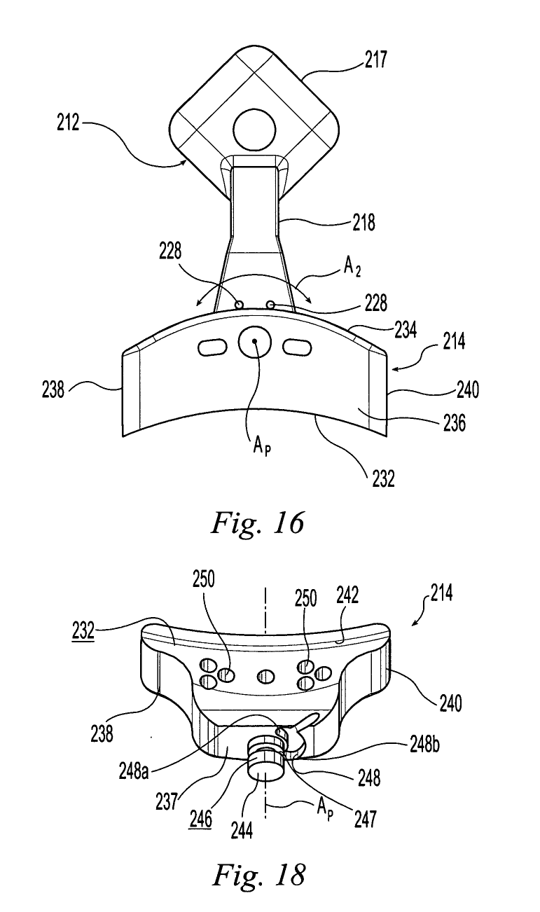

Turning now to FIGS. 14-18 tibial cut guide assembly 210 according to one embodiment of the present invention will now be described. Tibial cut guide assembly is adapted to be used with an assembly of other components including a tibial tubercle alignment bar, tibial boom, and a tibial depth gauge (not shown) such as those illustrated in U.S. Patent Publication 2004/0153066, which was herein incorporated by reference above. Tibial cut guide assembly 210 generally includes cut guide support member 212, tibial cut guide 214 pivotally coupled to support member 212, and vertical fixation member 216 securing cut guide 214 to support member 212.

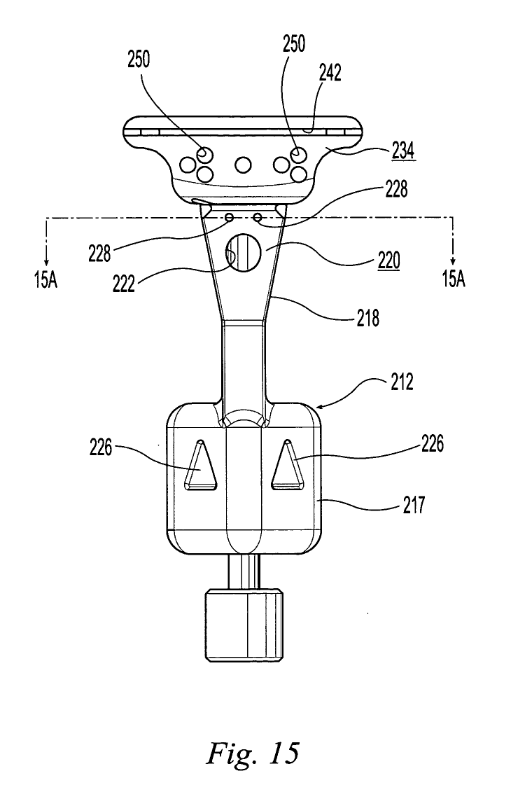

Referring particularly to FIGS. 14-16 and 18, support member 212 includes base 217 and leg 218 extending from base 217. Base 217 includes aperture 226 extending therethrough and configured to receive the extension bar (not shown) of a tibial boom (not shown) as shown in U.S. Patent Publication No. 2004/0153066. Leg 218 includes upper surface 219 and lower surface 220. Opening 222 extends through leg 218 from upper surface 219 to lower surface 220. Stop post 224 extends vertically from upper surface 219 of leg 218. Pin holes 228 extend through leg 218 at an angle to opening 222. As shown in FIG. 15A, pin holes 228 intersect opening 222 at diametrically opposed locations proximal the perimeter of opening 222.

Turning now to FIGS. 14-18, tibial cut guide 214 includes first bone engaging surface 232 and opposite second surface 234. Opposing proximal and distal sides 236, 237 extend between first and second surfaces 232, 234. Opposing medial and lateral ends 238, 240 extend between both first and second surfaces 232, 234 and proximal and distal sides 236, 237. Cut guide slot 242 extends through tibial cut guide 214 from first surface 232 to second surface 234 and provides cut guide surface 243. Cut guide slot 242 is configured to receive a cutting instrument (not shown) therethrough and guide surface 243 is adapted to guide the cutting instrument in resecting the proximal end of the tibia (not shown). Fastener receiving holes 250 extend through tibial cut guide 214 from first surface 232 to second surface 234. Fastener receiving holes 250 are adapted to receive fasteners therethrough such as pins, screws or nails.

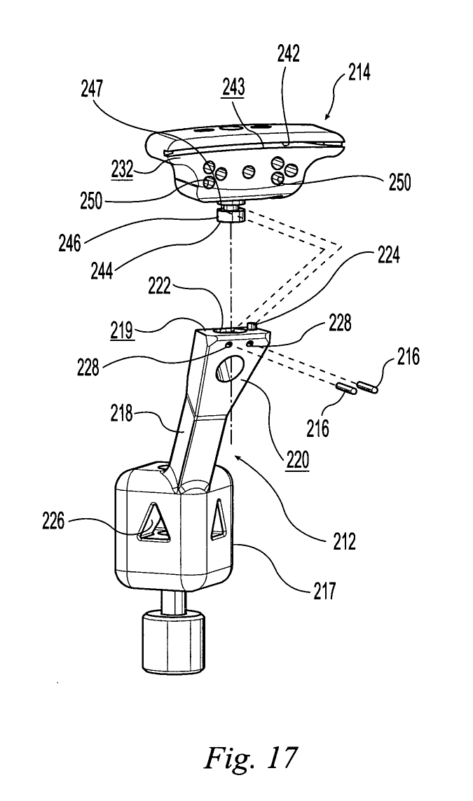

Referring to FIGS. 17 and 18, mounting post 244 extends vertically from distal side 237 of tibial cut guide 214 and defines post axis A_{P}. Mounting post 244 is substantially cylindrical and is defined by sidewall 246. Groove 247 is defined in sidewall 246 and extends about mounting post 244. Mounting post 244 is rotatably received in opening 222 to couple tibial cut guide 214 to support member 212. Accordingly, cut guide 214 is pivotal about post axis A_{P} relative to support member 212 in the direction of double-headed arrow A₂ and along a plane coplanar with cut guide slot 242. When mounting post 244 is positioned in opening 222, groove 247 in mounting post 244 is aligned with pin holes 228. Track 248 is defined in distal side 237 and extends along a semi-circular path centered about post axis A_{P}. Track 248 extends between first track end 248a and second track end 248b. Stop post 224 of support member 212 is received in and travels along track 248 between first and second track ends 248a, 248b. Stop post 224 cooperates with track 248 to limit the rotational movement of mounting post 244 in opening 222. In other words, when stop post 224 reaches either of first or second track ends 248a-b, further rotation of post 244 in opening 222 is prohibited. It should be noted that track 248 and stop post 224 need not be disposed on cut guide 214 and support member 212, respectively. Rather, track 248 and stop post 224 may be reversely positioned on support member 212 and cut guide 214, respectively.

Turning now to FIGS. 15A and 17, vertical fixation member 216 is in the form of a pair of pins sized and configured to fit into pin holes 228 of support member 212. Vertical fixation pins 216 extend through pin holes 228 and intersect opening 222 such that a central portion of pins 216 are disposed in groove 247, and thereby prevent mounting post 244 from moving vertically in opening 222 while permitting mounting post 244 to rotate within opening. The engagement of pins 216 in groove 247 prevents mounting post 216 from disengaging from opening 222.

In use, tibial cut guide assembly 210 is mounted to the extension bar (not shown) of a known tibial boom (not shown) such as that illustrated in U.S. Publication No. 2004/0153066 by inserting the extension bar through aperture 226. Aperture 226 is illustrated as having a triangular cross section to receive a triangular shaped extension bar. However, aperture 226 may be alternatively configured to receive an extension bar of different shapes, such as semi-circular. Cut guide 214 is positioned medially-laterally along the extension bar (not shown) and proximally-distally along the alignment bar (not shown) as is described in U.S. Publication No. 2004/0153066. Cut guide 214 is then pivoted about post axis A_{P} to achieve more specific placement and alignment of guide 214 against the tibia. The pivoting feature of tibial cut guide assembly 210 also assists in positioning and advancing cut guide 214 through the soft tissue to the surface of the tibia, thereby minimizing the surgical space and visibility needed. Once cut guide 214 is properly positioned against the tibia, fasteners (not shown) may be inserted through fastener receiving holes 250 and into the tibia to secure cut guide 214 to the tibia. Then a cutting instrument (not shown), such as a saw, may be inserted through cut guide slot 242 and into the tibia to resect the proximal end of the tibia.

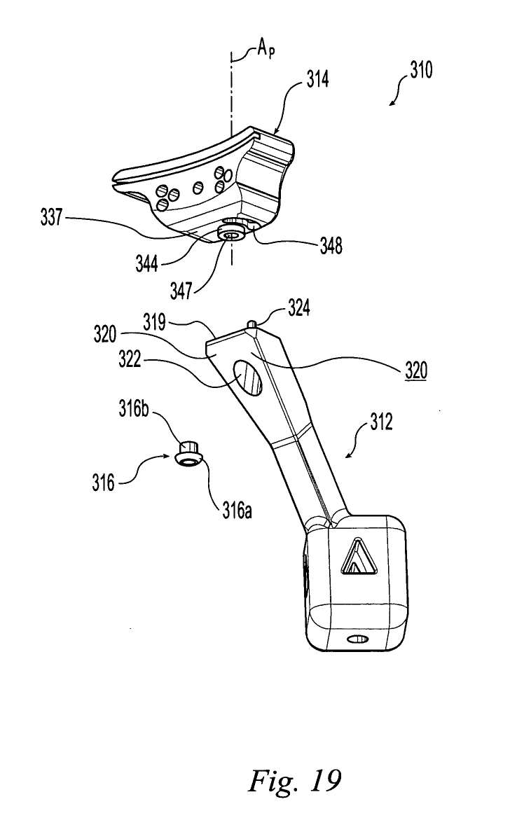

Although the embodiment described above discloses the vertical fixation member as a pair of pins, the present invention may be adapted to include only a single pin. Furthermore, vertical fixation of the mounting post may be achieved using other types of vertical fixation members. For example, turning to FIG. 19, tibial cut guide assembly 310 according to another embodiment of the present invention is illustrated. Tibial cut guide assembly 310 includes cut guide support member 312, cut guide 314 and vertical fixation member 316. Support member 312 includes upper surface 319 and lower surface 320. Opening 322 extends through support member from upper surface 319 to lower surface 320. Stop post 324 extends vertically from upper surface 319. Cut guide 314 includes distal surface 337 from which mounting post 344 extends. Mounting post 344 defines post axis A_{P} and is rotatably received in opening 322 of support member 312. Mounting post 344 includes central bore 347 extending therein along post axis A_{P}. Track 348 is defined in distal side 337 and extends along a semi-circular path having post axis A_{P} as its center. Stop post 324 is slidably received in track 348 to limit the pivoting of cut guide 314 relative to support member 312.

Vertical fixation member 316 includes enlarged head 316a and shaft 316b. Shaft 316b extends into opening 322 from lower surface 320 and is securely received within central bore 347, such as by a threaded or press-fit engagement. Enlarged head 316a is sized too large to pass through opening 322 and, thus, prevents mounting post 344 from moving vertically within, and disengaging from, opening 322. Opening 322 may be larger proximal lower surface 320 such that enlarged head 316a may be received within a lower portion of opening 322 and rotate therein. However, opening 322 proximal upper surface 319 is smaller in diameter than enlarged head 316a to prevent head 316a from passing through opening 322. Cut guide assembly 310 operates in substantially the same way as cut guide assembly 210 described above.

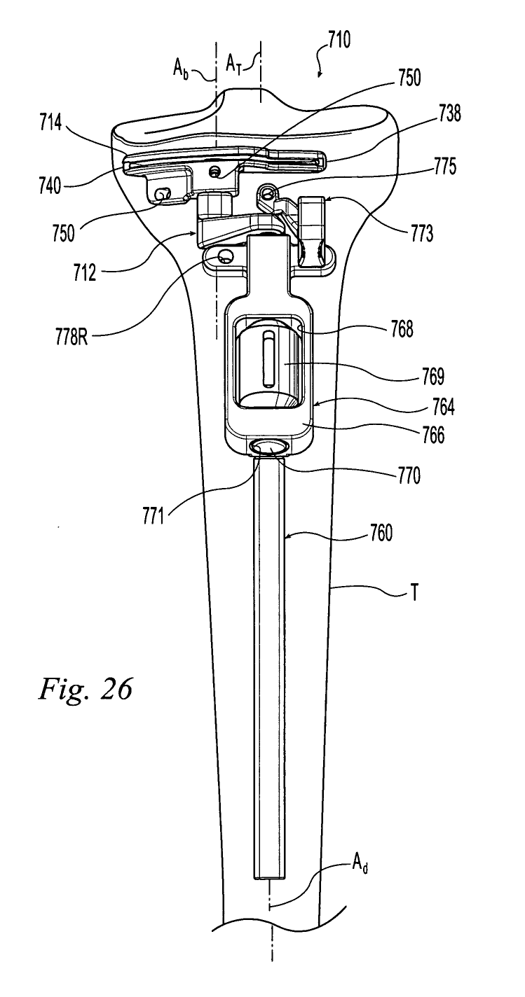

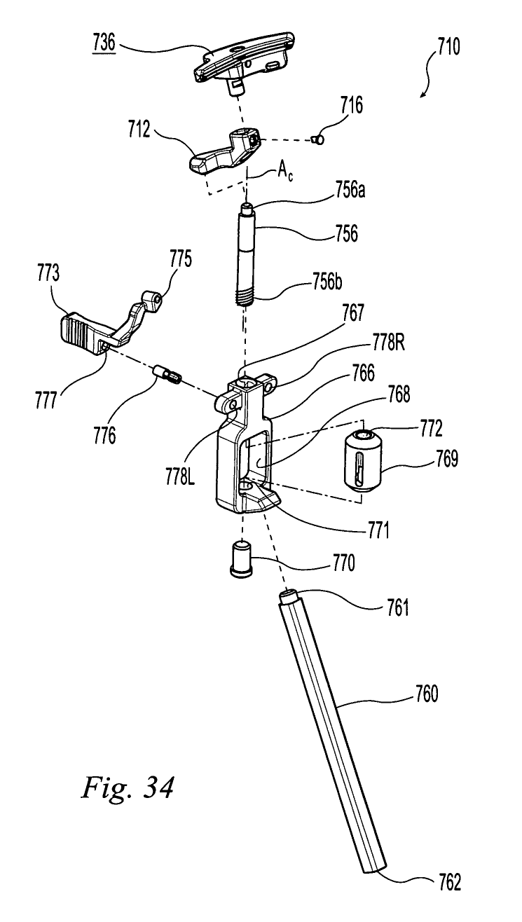

Referring now to FIGS. 26-36, tibial cut guide assembly 710 according to another embodiment of the present invention will now be described. As illustrated in FIGS. 33 and 34, tibial cut guide assembly 710 generally includes cut guide support member 712, tibial cut guide 714 pivotally coupled to support member 712 and vertical fixation member 716 securing cut guide 714 to support member 712.

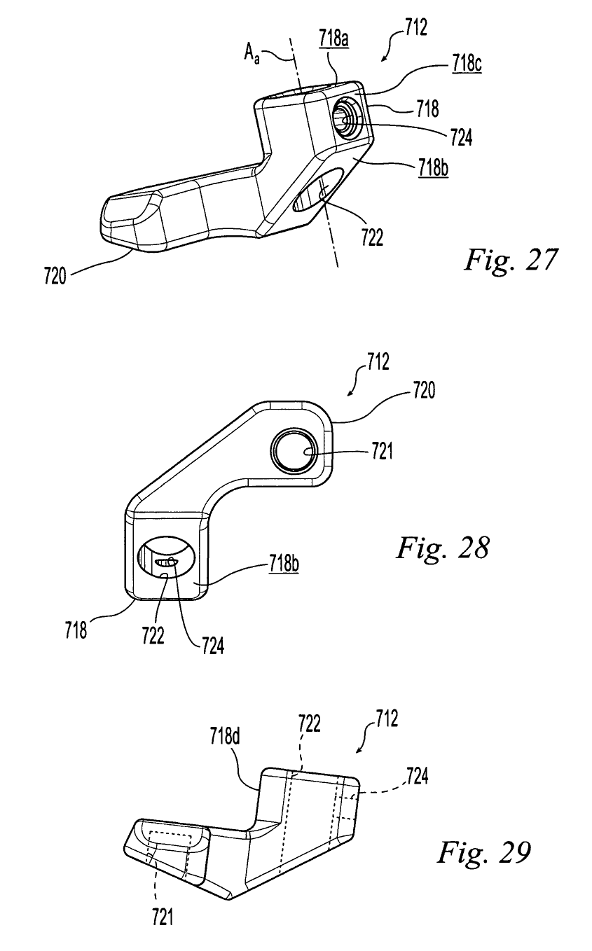

Referring particularly to FIGS. 27-29, support member 712 includes first end 718 and second end 720. First end 718 includes upper surface 718a, lower surface 718b and bone facing surface 718c. Support member 712 includes post receiving opening 722 extending through first end 718 from upper surface 718a to lower surface 718b and defining axis Aₐ. Hole 724 extends into bone facing surface 718c of first end 718 and intersects opening 722. Second end 720 of support member 712 includes stem receiving opening 721 extending into secured end 720.

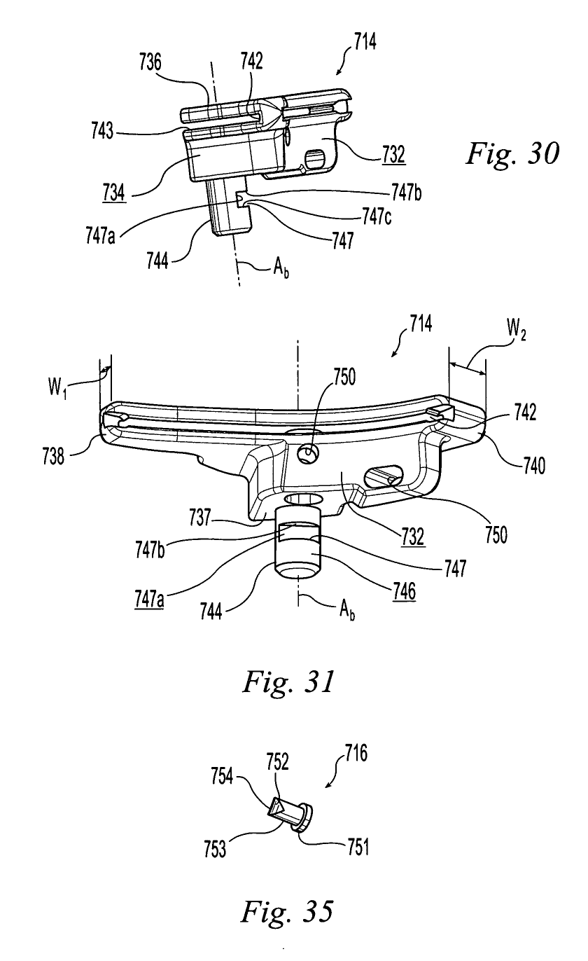

Turning now to FIGS. 30 and 31, tibial cut guide 714 includes bone engaging first surface 732 and opposing second surface 734. Bone engaging first surface 732 is contoured for placement against the surface of the tibia T, as is discussed in further detail below. Opposing proximal and distal sides 736, 737 extend between first and second surfaces 732, 734. Opposing medial and lateral ends 738, 740 extend between both first and second surfaces 732, 734 and proximal and distal sides 736, 737. As illustrated in FIG. 31, cut guide 714 has width W₁ extending between first and second surfaces 732, 734 at medial end 738. Cut guide 714 also has width W₂ extending between first and second surfaces 732, 734 at lateral end 740. Width W₁ is smaller than width W₂ to facilitate positioning of cut guide 714, as discussed in further detail below.

Referring still to FIGS. 30 and 31, cut guide 714 includes cut guide slot 42 extending through cut guide 714 from first surface 732 to second surface 734 and sized to receive a cutting instrument, such as a saw. Cut guide slot 742 is defined, in part, by cut guide surface 743 which provides a surface for guiding the cutting instrument. Fastener receiving holes 750 extend through cut guide 714 from first surface 732 to second surface 734 and are configured to receive a fastener, such as a nail, pin or screw, for fixing guide 714 to tibia T. As illustrated in FIGS. 26 and 31, holes 750 may have various shapes. For instance, holes 750 may have an elongated shape to allow for some adjustability in the positioning of the guide after the fastener is received therethrough. As shown in FIGS. 30 and 31, cut guide 714 also includes mounting post 744 extending from distal side 737 and defining post axis A_{b}. Mounting post 744 is defined by cylindrical sidewall 746 which extends about post axis A_{b}. Notch 747 cuts into sidewall 746 and, unlike groove 247 (FIGS. 14-18) in the previously discussed embodiments, extends about only a portion of sidewall 746. Notch 747 is defined by flat surface 747a, upper surface 747b and opposing lower surface 747c.

Turning now to FIG. 35, vertical fixation member in the form of pin 716 includes head 751 and shaft 753 extending from head 751. Shaft 753 is sized to be received in hole 724 of support member 712. Shaft 753 is beveled at end 755 opposite head 751 to provide beveled surface 752 and vertex 754.

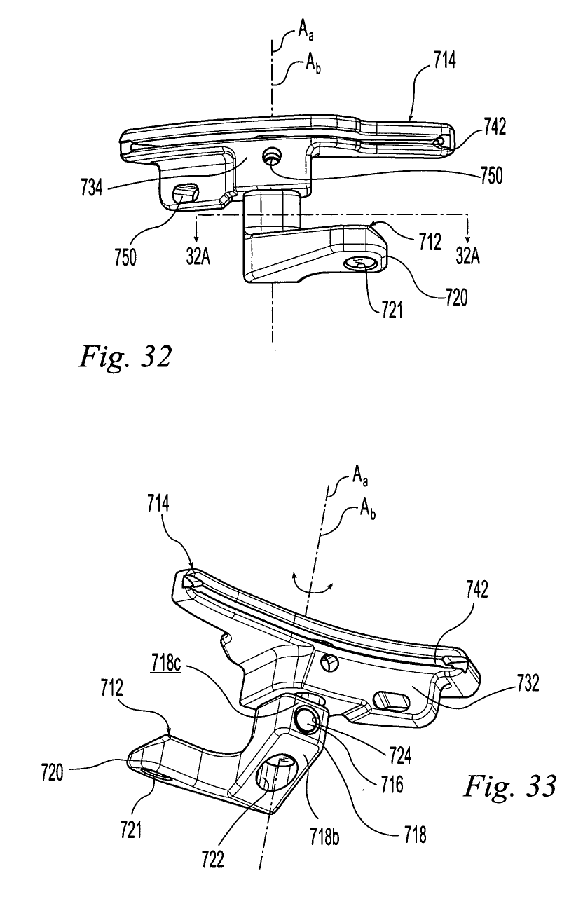

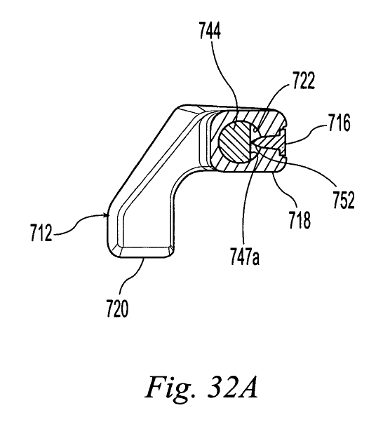

Referring now to FIGS. 30-34, cut guide 714 is pivotally mounted on support member 712 by inserting mounting post 744 of cut guide 714 into post receiving opening 722 of support member 712. When post 744 is received in opening 722, axes Aₐ and A_{b} are aligned with one another, as illustrated in FIGS. 32 and 33, and post 744 is rotatable within opening 722 about axes Aₐ and A_{b}. Shaft 753 of vertical fixation pin 716 extends through hole 724 of support member 712, and end 755 of shaft 753 extends into notch 747 of guide 712. End 755 of vertical fixation pin 716 cooperates with upper and lower surfaces 747b, 747c to restrict vertical movement of post 744 in opening 722. Vertex 754 of vertical fixation pin 716 is positioned proximal to or against flat surface 747a of notch 747, as shown in FIG. 32A. As post 744 rotates in opening 722 and 755 of pin 716 pivots within notch 747 such that flat surface 747a of notch 747 pivots about vertex 754 of pin 716. When beveled surface 752 on either side of vertex 754 reaches flat surface 747a of notch 747, further rotation of post 744 in that direction within opening 722 is prohibited.

FIGS. 27-31 illustrate notch 747 and hole 724 as positioned proximal bone engaging first surface 732 of cut guide 714 and bone facing surface 724 of support member 712, respectively. It should be understood that notch 747 and hole 728 may be disposed in any position provided that notch 747 cooperates with pin 716 in hole 724 to permit and limit the rotational post 744 in opening 722. For instance, notch 747 may be disposed in sidewall 746 proximal second surface 734, while hole 724 may extend into surface 718d of guide 712.

Referring now to FIGS. 26, 34 and 36, the assembly of cut guide 714, support member 712 and vertical fixation pin 716 shown in FIGS. 32 and 33 is adapted for use with riser stem 756, alignment rod member 760 and stem height adjustment member 764. Riser stem 756 extends between primary end 756a and secondary end 756b and defines shaft axis A_{c}. Primary end 756a is configured to fit within stem receiving opening 721 (FIG. 28) in support member 712 to thereby secure support member 712 to riser stem 756. Secondary end 756b is threaded.

Referring still to FIGS. 26, 34 and 36, stem height adjustment member 764 generally includes housing 766 and adjustment knob 769. Housing 766 defines chamber 768 configured to rotatably receive knob 769. Housing 766 includes stem receiving opening 767 and peg hole 771, each of which intersect chamber 768. Stem receiving opening 767 is configured to slidably receive stem 756. Peg hole 771 is adapted to receive peg 770. Housing 766 also includes pin openings 778 adapted to receive pin 776 as described in further detail below. Housing 766 also includes an alignment rod member receiving opening (not shown) for receiving an end of alignment rod member 760, as discussed in further detail below.

Referring still to FIGS. 26, 34 and 36, knob 769 is rotatably held within chamber 768 by peg 770, which extends through peg hole 771 in chamber 768 and into an opening (not shown) in knob 769. Secondary end 756b of riser stem 756 extends through stem opening 767 in housing 766 and threadedly engages threaded hole 772 in knob 769.

Alignment rod member 760 extends between first end 761 and opposing second end 762 and defines alignment axis A_{d}. First end 761 is adapted to fit within alignment member receiving opening (not shown) in housing 766 to thereby couple alignment rod member 760 to adjustment member 764. Second end 762 of alignment member 764 is configured to couple with any known ankle clamp (not shown) adapted to attach to a patient's ankle, as illustrated in The Zimmer Institute Surgical Technique, “MIS™ Quad-Spacing™ Surgical Technique for Total Knee Arthroplasty NEXGEN® COMPLETE KNEE SOLUTION,” The Zimmer Institute, 2004; Zimmer® MIS™ Tibial Cut Guide Assembly Surgical Technique Addendum, 2005; and in U.S. Patent Publication No. 2004/0102785, filed on Nov. 27, 2002 in the names of Hodorek et al. and entitled METHOD AND APPARATUS FOR ACHIEVING CORRECT LIMB ALIGNMENT IN UNICONDYLAR KNEE ARTHROPLASTY, each of which are hereby incorporated by reference.

Tibial cut guide assembly 710 also includes optional anchor member 773, which has pin hole 777 and fastener receiving hole 775. Pin hole 777 receives an end of pin 776, the opposite end of which is received in hole 778L of adjustment member 764, to couple anchor member 773 to adjustment member 764. Fastener receiving hole 775 is configured to receive a fastener, such as a nail screw or pin that may be fastened to tibia T.

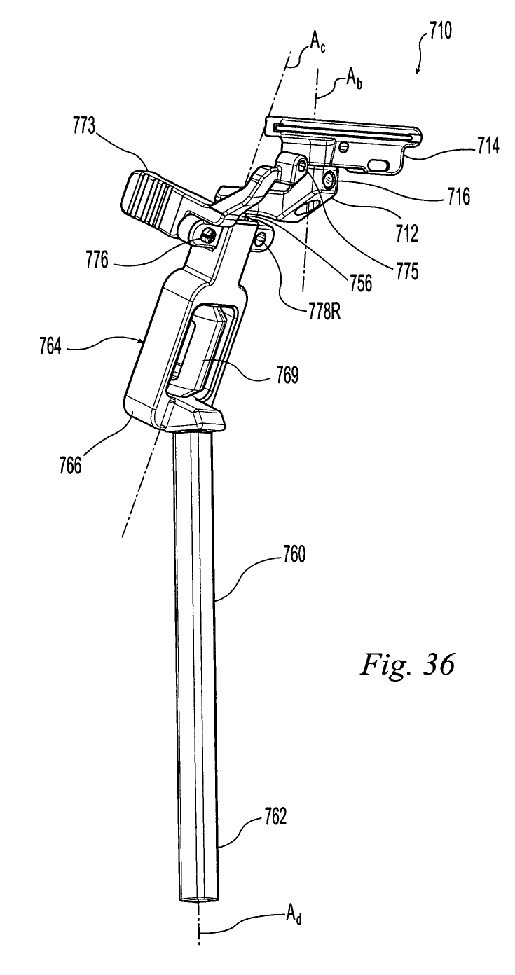

Referring to FIGS. 26 and 36, use of tibial cut guide assembly 710 will now be described. First, second end 762 of alignment member 760 is slidable coupled to an ankle bracket or clamp (not shown) as illustrated in The Zimmer Institute Surgical Technique, “MIS™ Quad-Spacing™ Surgical Technique for Total Knee Arthroplasty NEXGEN® COMPLETE KNEE SOLUTION,” The Zimmer Institute, 2004; Zimmer® MIS™ Tibial Cut Guide Assembly Surgical Technique Addendum, 2005; and U.S. Patent Publication No. 2004/0102785, incorporated by reference above.

The ankle bracket is then attached to the patient's ankle and alignment rod member is positioned such that alignment axis A_{d} is aligned with mechanical axis A_{T} of Tibia T. Alignment rod member 760 may be secured in this position by inserting a fastener (not shown), such as a nail, screw or pin, through fastener hole 775 in anchor member 773 and into tibia T. Optional anchor member 773 provides added stability in the position of alignment rod member 760 while the position of cut guide 714 is adjusted. Next, the height of cut guide 714 (i.e. the depth of the proximal cut) may be adjusted by rotating knob 769 of adjustment member 764. As knob 769 is rotated, threaded secondary end 756b (FIG. 34) of riser shaft 756 moves further into or out of threaded engagement with threaded opening 772 (FIG. 34) of knob 769. As a result, riser shaft 756 slides proximally/distally along shaft axis A_{c}, thereby raising or lowering support member 712 and cut guide 714 relative to adjustment member 764. As illustrated in FIG. 36, shaft axis A_{c} extends at an angle to alignment axis A_{d}. Because alignment axis A_{d} is aligned with mechanical axis A_{T} of tibia T, shaft axis A_{c} extends at an angle to mechanical axis A_{T}. As a result, movement of riser shaft 756 along shaft axis A_{c} also moves cut guide 714 toward or away from tibia T.

At any point during the positioning of cut guide 714 relative to tibia T, cut guide 714 may be rotated about post axis A_{b} to facilitate positioning of cut guide 714 and advancement of cut guide 714 through soft tissue and under the patella (not shown). In addition, as shown in FIG. 31, medial end 738 of cut guide 714 has reduced width W₁ relative to width W₂ of lateral end 740. This reduced width W₁ facilitates the insertion and positioning of end 738 under the patella (not shown), patella tendon, quad tendon and other tissues. Once cut guide 714 is in the desired position, cut guide 714 may be secured to tibia T by inserting fasteners (not shown) such as nails, screws or pins, through fastener receiving holes 750 and into tibia T.

Cut guide 714 and support member 712, as illustrated, are configured for use in a medial approach of the left knee. It should be understood that tibial cut guide assembly 710 may be adapted for use in a medial approach of the right knee by forming a mirror image of support member 712, cut guide 714 and anchor member 773. In this case, anchor member 773 would be mounted to opening 778R on the other side of housing 766 via peg 776. It should also be understood that cut guide 714 could be adapted for use with a support member of a different design, such as support member 212 described above and shown in FIGS. 14-16.

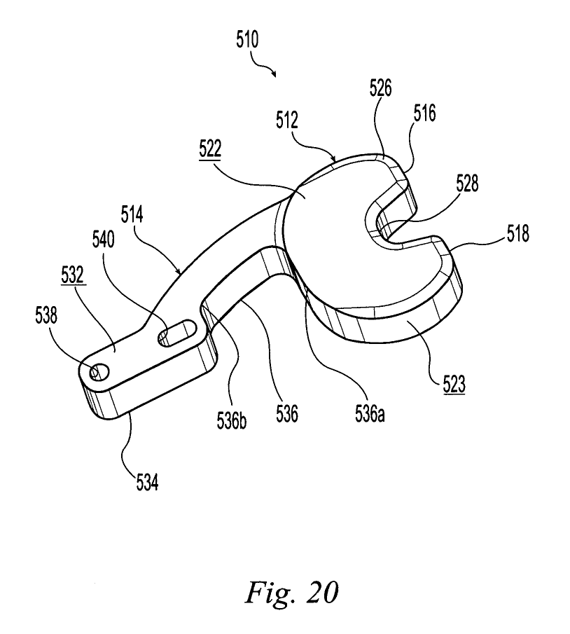

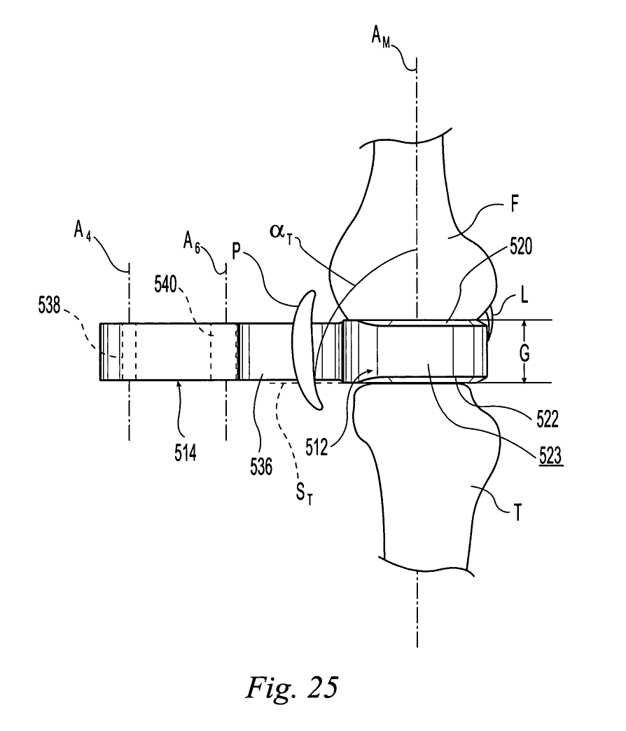

Turning now to FIGS. 20-25, exemplary arthroplastic spacer apparatus 510 according to one embodiment of the present invention will now be described. Spacer apparatus 510 generally includes spacer block 512 and handle 514 extending from spacer block 512. Spacer block 512 is configured to gauge gap G (FIG. 25) between resected distal end of femur F and resected proximal end of tibia T. Spacer block 512 may be made from any firm surgical grade material, including surgical stainless steel. Spacer block 512 includes medial side 515, lateral side 517 opposite medial side 515, anterior side 519, and posterior side 521 opposite anterior side 519. Spacer block 512 also includes opposing superior and inferior gauge surfaces 520, 522 extending between medial, lateral, anterior and posterior sides 515, 517, 519, 521. Perimeter surface 523 extends between superior and inferior surfaces 520, 522 and wraps both anteriorly-posteriorly and medially-laterally about the perimeter of spacer block 512. Superior and inferior gauge surfaces 520, 522 are substantially smooth and planar and are configured to slide against distal end of femur F and proximal end of tibia T, respectively, without significantly abrading or otherwise damaging nearby tissues. Similarly, perimeter surface 523 is substantially smooth and is configured to slide against soft tissues such as muscle, cartilage, ligaments, and the like without significantly cutting, tearing, or otherwise damaging the tissues. A portion of the edge joining superior surface 520 and perimeter surface 523 is beveled (beveled superior edge 524), while a portion of the edge joining inferior surface 522 and perimeter surface 523 is also beveled (beveled inferior edge 526).

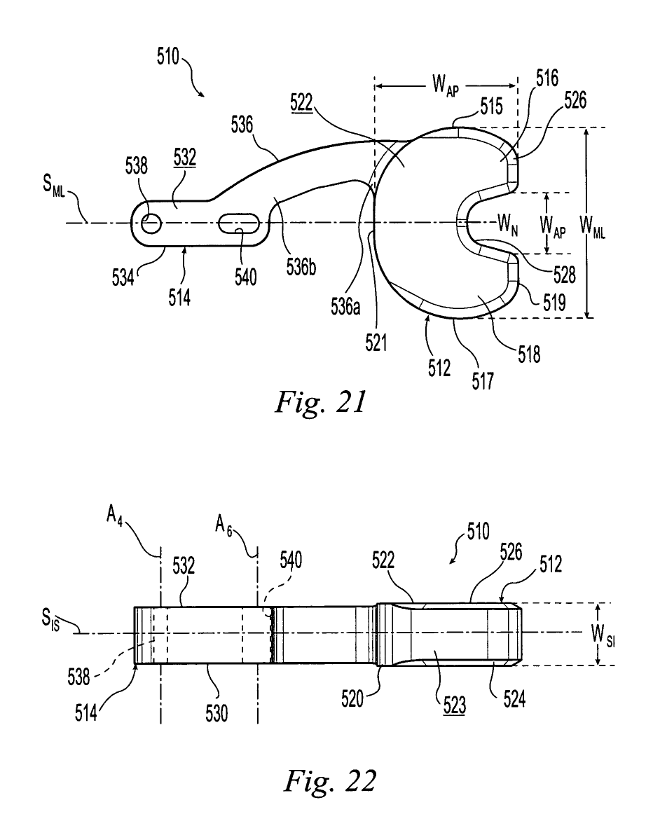

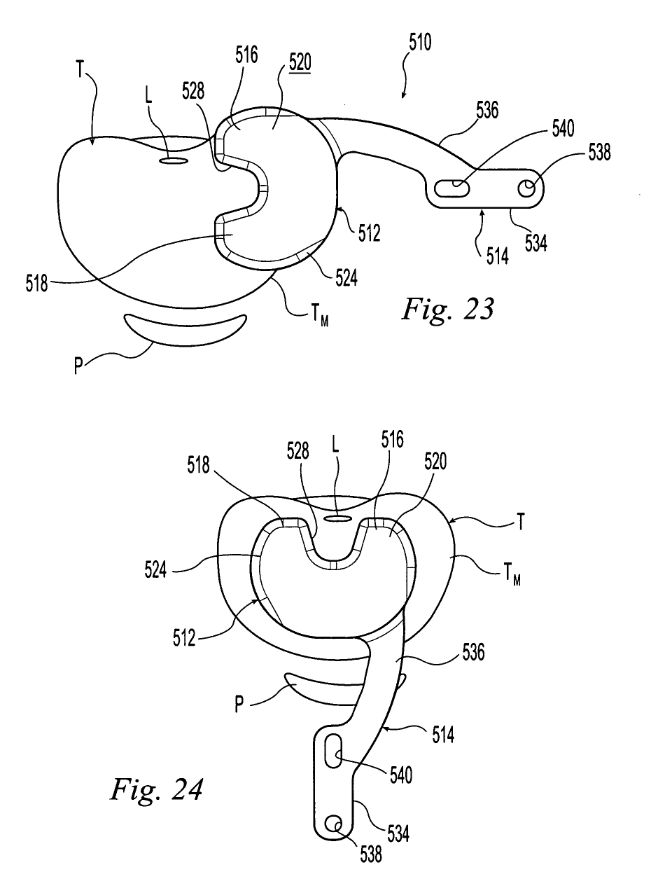

Referring now to FIGS. 21, 22 and 25, spacer block 512 is substantially symmetrical and includes a medial portion or medial lobe 516 and a lateral portion or lateral lobe 518. Spacer block 512 has a medial-lateral width W_{ML} extending between medial and lateral sides 515, 517. An axis of symmetry or split-plane S_{ML} divides medial-lateral width W_{ML} and separates medial lobe 516 from lateral lobe 518. Spacer block 512 also has an anterior-posterior width W_{AP} extending between anterior and posterior sides 519, 521. Spacer block 512 includes generally U-shaped posterior notch 528 between medial lobe 516 and lateral lobe 518. As is discussed in further detail below, notch 528 is configured to arc around posterior cruciate ligament L (FIG. 23-25) during operation of spacer apparatus 510. Notch 528 is centered about split-plane S_{ML} and has a medially-laterally extending notch width W_{N}, which is about one-third as large as medial-lateral width W_{ML}. Spacer block also has an superior-inferior width W_{SI} extending between superior surface 520 and inferior surface 522.

Referring to FIGS. 20-22 and 25, handle 514 includes linear portion 534 and curved portion 536 coupling linear portion 534 to spacer block 512. Curved portion 536 includes first end 536a and second end 536b. First end 536a of curved portion 536 extends from perimeter surface 523 of spacer block 512 at a point medial to split-plane S_{ML}. Second end 536 of curved portion 536 is coupled to linear portion 534 such that linear portion 534 is aligned with and is longitudinally bisected by split-plane S_{ML}. Curved portion 536 curves medially away from split-plane S_{ML} such that handle 514 is configured to arc around non-everted or naturally positioned patella P (FIG. 23-25) during operation of apparatus 510, as discussed in further detail below.

Linear portion 534 of handle 514 includes upper surface 530 and lower surface 532. Cylindrical hole 538 extends through linear portion 534 from upper surface 530 to lower surface 532 along opening axis A₄. Axis A₄ intersects split-plane S_{ML} and, as discussed in further detail below, hole 538 is configured such that axis A₄ is parallel to mechanical axis A_{M} of femur F (FIG. 25) when spacer block 512 is positioned in gap G (FIG. 25). Elongated slot 540 extends through linear portion 534 from upper surface 530 to lower surface 532 along opening axis A₆. Similar to axis A₄, axis A₆ intersects split-plane S_{ML} and, as discussed in further detail below, slot 540 is configured such that axis A₆ is parallel to mechanical axis A_{M} of femur F (FIG. 25) when spacer block 512 is positioned in gap G (FIG. 25).

Although in the exemplary embodiment described above linear portion 534 is straight and curved portion 536 is curved, in alternative embodiments linear portion 534 and/or curved portion 536 of handle 514 may be straight, piecewise linear, curvilinear, or of any other suitable geometry such that a portion of curved portion 536 is positioned medially outwardly of split-plane S_{ML} to avoid impingement of patella P during operation of apparatus 510. Similar to spacer block 512, handle 514 may be made from any surgical grade material including surgical stainless steel. Handle 514 may be integrally formed as a single unit with spacer block 512. Alternatively, handle 514 may be a component discrete from and attachable to spacer block 512.

Referring now to FIGS. 23-25, operation of spacer apparatus 510 to gauge gap G between the resected distal end of femur F and the resected proximal end of tibia T will now be described. Prior to inserting spacer block 512 into gap G a suitable incision is made along the medial side of knee, and then distal end of femur F and proximal end of tibia T are resected in a known manner to provide gap G. The distal end of femur F and proximal end of tibia T may be resected to accommodate or correct the difference between the anatomic axis (an imaginary line drawn down the center of the femoral canal) and the mechanical axis (a line passing through the center of the hip, the center of the knee and the center of the ankle). In this exemplary embodiment, that difference is about 7°. The surgeon may employ minimally invasive surgical techniques to make the resections without everting patella P.

Next, by grasping and manipulating handle 514, spacer apparatus 510 is positioned such that split-plane S_{ML} is aligned in a medially-laterally direction relative to the knee, as shown in FIG. 23. Spacer block 512 is then inserted in a lateral direction into gap G. The smooth configuration of superior and inferior beveled edges 524, 526, superior and inferior surfaces 520, 522 and perimeter surface 523 facilitates the insertion of spacer block 512 without damage to any soft tissues (not shown). Next, by grasping and manipulating handle 514 the surgeon rotationally translates spacer apparatus 512 by about 90 degrees such that apparatus 512 is moved into the space gauging position shown in FIGS. 24 and 25. The smooth configuration of superior and inferior beveled edges 524, 526, superior and inferior surfaces 520, 522 and perimeter surface 523 also facilitates the rotation of spacer apparatus 510 without damage to any nearby soft tissues (not shown). In addition, notch 528 curves around posterior cruciate ligament L thereby avoiding damage to posterior cruciate ligament L. Furthermore, curved portion 536 of handle 514 arcs around patella P to avoid impingement of patella P.

FIG. 25 illustrates spacer apparatus 510 being used to gauge gap G (between distal femur F and proximal tibia T). When proximal end of tibia T and distal end of femur F have been resected to correct the difference between the anatomic axis and the mechanical axis, slope angle α_{T} (i.e. the angle between the mechanical axis A_{M} and the plane of the resected tibial surface S_{T}) is about 83° relative to the mechanical axis A_{M} (accommodating the 7° difference). Thus, when superior and inferior surfaces 520, 522 are positioned against the resected end of femur F and the resected end of tibia T, respectively, axes A₄ and A₆ are positioned roughly parallel to mechanical axis A_{M}. Therefore, a rod or other suitable alignment apparatus (not shown) may be inserted through hole 538 and/or slot 540 for assessment or verification of angle α_{T} in a known manner. More particularly, the position of the rod may be compared to mechanical axis A_{M} to check the tibial slope and verify the proper varus and valgus alignment. If the rod inserted into hole 538 or slot 540 is in parallel alignment with mechanical axis A_{M} then proper tibial slope and varus/valgus alignment has been achieved. In addition, after positioning apparatus 510 as discussed, gap G may be gauged by visually comparing it to superior-inferior width W_{SI}.

Exemplary arthroplastic spacer apparatus 510 is illustrated and described for use in a medial approach application (entering from the medial side of the knee). It should be understood that the arthroplastic spacer apparatus of the present invention may be adapted for use in a lateral approach application (entering from the lateral side of the knee), simply by making a mirror-image of arthroplastic spacer apparatus 510.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A tibial cut guide assembly for cutting a proximal end of the tibia bone, the tibia bone defining a tibial axis and including the proximal end and an anterior surface, the cut guide assembly comprising:
a cut guide support member having a first end and a second end, said first end having an upper surface and a lower surface, said support member having an opening extending through said first end from said upper surface to said lower surface, said opening defining a first axis, said support member having a hole extending into said first end and intersecting said opening;
a cut guide having a first bone engaging surface, an opposing second surface, and opposing proximal and distal sides extending between said first and second surfaces, said cut guide having at least one cut guide surface extending from said first surface to said second surface, said cut guide having a mounting post extending vertically from said distal side, said mounting post having a sidewall extending about said first axis and a notch defined in and extending about a portion of said sidewall, said mounting post rotatably received within said opening of said support member such that said notch is aligned with said hole; and
a pin extending through said hole, said pin having a first end extending into said notch to prevent vertical movement of said mounting post along said first axis, said first end of said pin pivotal within said notch to permit and limit rotation of said mounting post about said first axis.

2. The tibial cut guide assembly of claim 1 wherein said notch includes a flat surface extending along a plane parallel to said first axis.

3. The tibial cut guide assembly of claim 1 where in said first end of said pin is beveled.

4. The tibial cut guide assembly of claim 1 wherein said bone engaging first surface is contoured for placement against the anterior surface of the tibia bone.

5. The tibial cut guide assembly of claim 1 wherein said cut guide surface includes an elongated slot extending through said cut guide from said first surface to said second surface.

6. The tibial cut guide assembly of claim 1 wherein said first end of said cut guide support member includes a first bone facing side and an opposite second side, said first and second sides extending between said upper and lower surfaces, said notch is disposed in said sidewall of said post proximal said first bone engaging surface, and said hole extending into said first bone facing side of said first end.

7. The tibial cut guide assembly of claim 1 wherein said first end of said cut guide support member includes a first bone facing side and an opposite second side, said first and second sides extending between said upper and lower surfaces, said notch is disposed in said sidewall of said post proximal said second surface, and said hole extending into said second side of said first end.

8. The tibial cut guide assembly of claim 1 wherein said cut guide assembly further includes:
an elongate riser stem defining a stem axis and having a primary end and an opposing secondary end, said primary end coupled to said second end of said cut guide support member;
an elongate alignment rod member defining an alignment axis and adapted to be mounted to the tibia with said alignment axis being parallel to the tibial axis; and
a stem height adjustment member coupled to both said secondary end of said riser stem and said alignment rod member, said stem height adjustment member operable to translate riser stem along said stem axis.

9. The tibial cut guide assembly of claim 1 wherein said cut guide has a medial end and an opposing lateral end, said medial and lateral ends extend between said opposing first and second surfaces and said opposing proximal and distal sides, said medial end defines a first width extending between said opposing first and second surfaces, said lateral end defines a second width extending between said opposing first and second surfaces, said first width is less than said second width.

10. A tibial cut guide assembly for cutting a proximal end of the tibia bone, the tibia bone including the proximal end and an anterior surface, the cut guide assembly comprising:
a cut guide support member having a first end and a second end, said first end having an opening extending therethrough and defining a first axis, said support member having a hole extending into said first end at an angle to said first axis and intersecting said opening;
a cut guide having a first bone engaging surface, an opposing second surface, and opposing proximal and distal sides extending between said first and second surfaces, said cut guide having at least one cut guide surface extending from said first surface to said second surface, said cut guide having a mounting post extending from said distal side, said mounting post having a vertical sidewall and a flat notch defined in said sidewall, said mounting post rotatably received within said opening of said support member such that said notch is aligned with said hole; and
a pin extending through said hole, said pin having a first end extending into said notch to prevent movement of said mounting post within said opening along said first axis, said first end of said pin pivotal within said notch to permit and limit rotation of said mounting post about said first axis.

11. The tibial cut guide assembly of claim 10 where in said first end of said pin is beveled.

12. The tibial cut guide assembly of claim 10 wherein said bone engaging first surface is contoured for placement against the anterior surface of the tibia bone.

13. The tibial cut guide assembly of claim 10 wherein said cut guide has a medial end and an opposing lateral end, said medial and lateral ends extend between said opposing first and second surfaces and said opposing proximal and distal sides, said medial end defines a first width extending between said opposing first and second surfaces, said lateral end defines a second width extending between said opposing first and second surfaces, said first width is less than said second width.

14. The tibial cut guide assembly of claim 10 wherein said first end of said cut guide support member has an upper surface, a lower surface, a first bone facing side and an opposite second side, said first and second sides extending between said upper and lower surfaces, said opening extending through said first end from said upper surface to said lower surface, said notch is disposed in said sidewall of said post proximal said first bone engaging surface, and said hole extending into said first bone facing side of said first end.

15. The tibial cut guide assembly of claim 10 wherein said first end of said cut guide support member has an upper surface, a lower surface, a first bone facing side and an opposite second side, said first and second sides extending between said upper and lower surfaces, said opening extending through said first end from said upper surface to said lower surface, said notch is disposed in said sidewall of said post proximal said second surface, and said hole extending into said second side of said first end.

16. The tibial cut guide assembly of claim 10 wherein said cut guide assembly further includes:
an elongate riser stem defining a stem axis and having a primary end and an opposing secondary end, said primary end coupled to said second end of said cut guide support member;
an elongate alignment rod member defining an alignment axis and adapted to be mounted to the tibia with said alignment axis being parallel to the tibial axis; and
a stem height adjustment member coupling said riser stem to said alignment rod member, said stem height adjustment member operable to translate riser stem along said stem axis.

17. The tibial cut guide assembly of claim 16 wherein said stem axis is at an angle relative to said first axis.

18. A tibial cut guide assembly for cutting a proximal end of the tibia bone, the tibia bone including the proximal end and an anterior surface, the cut guide assembly comprising:
a cut guide support member having a first end and a second end, said first end having an opening extending therethrough and defining a first axis, said support member having a hole extending into said first end at an angle to said first axis and intersecting said opening;
a cut guide having a first bone engaging surface, an opposing second surface, and opposing proximal and distal sides extending between said first and second surfaces, said cut guide having at least one cut guide surface extending from said first surface to said second surface, said cut guide having a mounting post extending from said distal side, said mounting post having a sidewall extending about said first axis and a notch defined in said sidewall and extending about a portion of said sidewall, said mounting post rotatably received within said opening of said support member such that said notch is aligned with said hole; and
a pin extending through said hole, said pin having a first end extending into said notch to prevent movement of said mounting post within said opening along said first axis, said first end of said pin movable within said notch to permit and limit rotation of said mounting post about said first axis.

19. The tibial cut guide assembly of claim 18 wherein said notch includes a flat surface extending along a plane parallel to said first axis.

20. The tibial cut guide assembly of claim 18 where in said first end of said pin is beveled.
